(19) United States
(12) Patent Application Publication
(10) Pub. No.: US 2025/0235178 A1
(43) Pub. Date: Jul. 24, 2025

(54) MEDICAL IMAGE PROCESSING APPARATUS AND X-RAY COMPUTED TOMOGRAPHY APPARATUS

(71) Applicant: CANON MEDICAL SYSTEMS CORPORATION, Otawara-shi (JP)

(72) Inventors: Ryusei SAIKI, Nasushiobara (JP); Hirona KUSUME, Nagoya (JP)

(73) Assignee: CANON MEDICAL SYSTEMS CORPORATION, Otawara-shi (JP)

(21) Appl. No.: 19/023,804

(22) Filed: Jan. 16, 2025

(30) Foreign Application Priority Data
Jan. 19, 2024 (JP) 2024-006617

(51) Int. Cl. A61B 6/00; A61B 6/03; G06T 7/70

(52) U.S. Cl. CPC A61B 6/547; A61B 6/032; G06T 7/70; G06T 2207/10081; G06T 2207/30004; G06T 2207/30196

(57) ABSTRACT

A medical image processing apparatus according to an exemplary embodiment includes processing circuitry. The processing circuitry determines a body position of a subject during a scan based on an imaging condition related to the scan of the subject. The processing circuitry identifies identify, from a storage device that stores a body position image representing the body position in association with a corresponding type of the body position, a body position image corresponding to the body position of the subject determined. The processing circuitry generates subject information about the subject in which the medical image obtained by the scan is in association with the body position image identified. The processing circuitry outputs the subject information generated.

### CROSS-REFERENCE TO RELATED APPLICATIONS

This application is based upon and claims the benefit of priority from Japanese Patent Application No. 2024-006617, filed Jan. 19, 2024, the entire contents of which are incorporated herein by reference.

### FIELD

Embodiments disclosed in the present specification and the accompanying drawings relate to a medical image processing apparatus and an X-ray computed tomography apparatus.

### BACKGROUND

There have been conventionally developed X-ray computed tomography apparatuses (hereinafter, referred to as X-ray CT apparatuses) that are capable of scanning a subject. In such X-ray CT apparatuses, it is possible to scan a subject in various body positions, such as standing position, sitting position, and lying position (hereinafter, also referred to as imaging modes), by changing the body posture of the subject or altering the shape of the gantry.

In an X-ray CT apparatus, the direction of gravity on a subject differs between when the subject is imaged in a standing or sitting position and when the subject is imaged in a lying position. Thus, the subject's organs are seen differently in medical images.

Therefore, a doctor needs to correctly grasp the body posture of the subject at the time of imaging and the imaging mode in which the subject was imaged in order to perform diagnosis. This is because the relative position between organs change due to the influence of the direction of gravity, and the organ to be diagnosed may be hidden behind another organ depending on the body posture of the subject at the time of imaging. In addition, the imaging mode may be included in the supplementary information of the medical image.

In an X-ray CT system including an X-ray CT apparatus, it is easy to determine the imaging mode of a medical image because the imaging mode can be checked on the X-ray CT apparatus. However, when a medical image is transferred to an external apparatus outside the X-ray CT system, imaging modes become mixed, and it may be difficult to determine the imaging mode of the medical image.

For example, the supplementary information of a medical image may be hard to see, or a tag containing the imaging mode cannot be displayed in a viewer outside the X-ray CT system, which makes it difficult to determine the imaging mode in some cases.

As an example, if a large number of medical images are displayed and the display size of the supplementary information is relatively small, the supplementary information may be hard to see and the imaging mode may be difficult to determine. Therefore, in the case of determining the imaging mode on a viewer outside the X-ray CT system, it may be difficult to determine the imaging mode, indicating room for improvement.

### BRIEF DESCRIPTION OF THE DRAWINGS

FIG. 1 is a diagram illustrating an example of a configuration of an X-ray CT apparatus according to an exemplary embodiment.

FIG. 2 is a diagram illustrating an example of a configuration of an X-ray CT system according to the exemplary embodiment.

FIG. 3 is a diagram illustrating an example of body position information according to the exemplary embodiment.

FIG. 4 is a diagram illustrating an example of subject information according to the exemplary embodiment.

FIG. 5 is a flowchart illustrating an example of a procedure of a control process of the X-ray CT apparatus according to the exemplary embodiment.

FIG. 6 is a diagram illustrating an example of medical information according to a seventh modified example.

### DETAILED DESCRIPTION

A medical image processing apparatus according to an exemplary embodiment includes processing circuitry. The processing circuitry determines a body position of a subject during a scan based on an imaging condition related to the scan of the subject. The processing circuitry identifies identify, from a storage device that stores a body position image representing the body position in association with a corresponding type of the body position, a body position image corresponding to the body position of the subject determined. The processing circuitry generates subject information about the subject in which the medical image obtained by the scan is in association with the body position image identified. The processing circuitry outputs the subject information generated.

Various Embodiments will be described hereinafter with reference to the accompanying drawings.

Hereinafter, exemplary embodiments of a medical image processing apparatus and an X-ray computed tomography apparatus (hereinafter, referred to as an X-ray CT apparatus) will be described with reference to the accompanying drawings. In the following exemplary embodiments, components with the same reference numerals perform similar operations, and thus duplicated descriptions thereof will be omitted as appropriate.

### Exemplary Embodiments

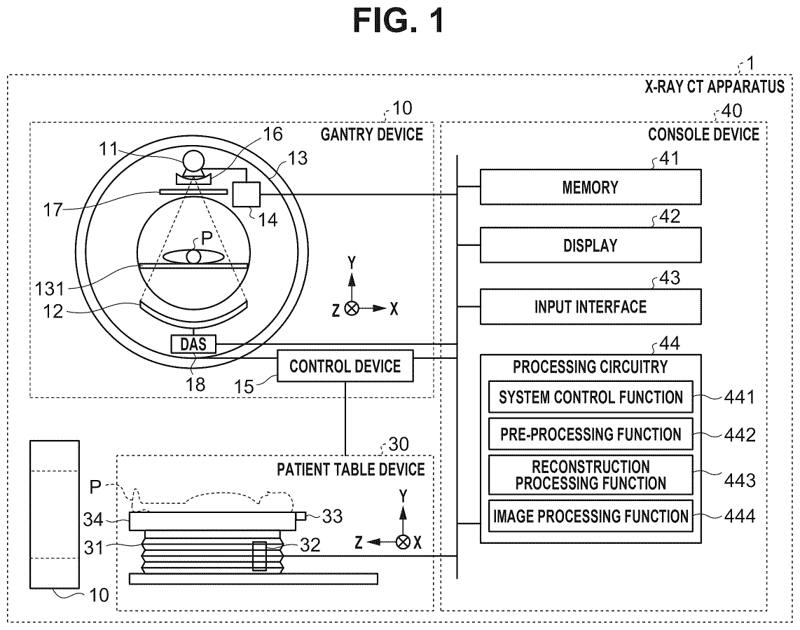

FIG. 1 is a diagram illustrating an example of a configuration of an X-ray CT apparatus 1 according to an exemplary embodiment. As illustrated in FIG. 1, the X-ray CT apparatus 1 includes gantry devices 10 (also referred to as gantries), a patient table device 30, and a console device 40.

In the present exemplary embodiment, the longitudinal direction of a rotation axis of a rotational frame 13 in a non-tilted state is defined as the Z-axis direction, the direction orthogonal to the Z-axis direction and toward the support column supporting the rotational frame 13 from the center of rotation is defined as the X-axis direction, and the direction orthogonal to the Z-axis and the X-axis is defined as the Y-axis direction. For convenience of description, a plurality of gantry devices 10 is illustrated in FIG. 1, but the actual configuration of the X-ray CT apparatus 1 includes only one gantry device 10.

The X-ray CT apparatus 1 illustrated in FIG. 1 has the patient table device 30 to allow a subject P in a lying position to be scanned. However, the X-ray CT apparatus 1 in the first exemplary embodiment may exclude the patient table device 30.

For example, in a case where the opening of the gantry device 10 in the X-ray CT apparatus 1 has a substantially cylindrical shape that extends vertically, the subject P will be scanned in a standing position, and therefore the patient table device 30 is not required. The X-ray CT apparatus in this case is called an upright CT apparatus.

In addition, the state of the gantry device 10 may be alterable, for example, the rotation axis of the rotational frame 13 may be alterable between the horizontal and vertical directions, such that the subject P can be scanned in any position, such as a lying position or a standing position. In this case, in accordance with a change in the state of the gantry device 10, the patient table device 30 is retracted during the standing position and the alteration, and is moved to the position illustrated in FIG. 1 during the lying position, for example.

Further, the state of the gantry device 10 may be alterable, for example, the rotation axis of the rotational frame 13 may be alterable between the horizontal and vertical directions, such that the subject P can be scanned in an oblique position in which the subject P is tilted obliquely relative to the horizontal plane. In this case, the patient table device 30 is tilted as appropriate without interfering with the gantry device 10 in accordance with a change in the state of the gantry device 10, for example. As described above, the X-ray CT apparatus 1 in the present exemplary embodiment may have any type of gantry device 10.

The gantry device 10 and the patient table device 30 operate based on operations performed by an operator via the console device 40, or via an operation unit provided in the gantry device 10 or the patient table device 30. The gantry device 10, the patient table device 30, and the console device 40 are connected each other by wire or wirelessly so as to be communicable with one another.

The gantry device 10 irradiates the subject P with X-rays and includes an imaging system that collects projection data from detection data on the X-rays having passed through the subject P. The gantry device 10 includes an X-ray tube 11, an X-ray detector 12, the rotational frame 13, an X-ray high-voltage device 14, a control device 15, a wedge 16, a collimator 17, and a data acquisition system (DAS) 18.

The X-ray tube 11 is a vacuum tube that generates X-rays by emitting thermoelectrons from a cathode (filament) toward an anode (target) through the application of a high voltage from the X-ray high-voltage device 14 and the supply of a filament current. X-rays are generated by the thermoelectrons colliding with the target. The X-rays generated at a tube focus of the X-ray tube 11 pass through an X-ray radiation window in the X-ray tube 11 and are shaped into, for example, a cone beam via the collimator 17, and are applied to the subject P. Examples of the X-ray tube 11 include a rotating anode X-ray tube that generates X-rays through irradiation of a rotating anode with thermoelectrons.

The X-ray detector 12 detects the X-rays that have been emitted from the X-ray tube 11 and passed through the subject P, and outputs an electrical signal corresponding to an amount of X-rays to the DAS 18. The X-ray detector 12 has a plurality of detection element arrays in which a plurality of detection elements is arranged in a channel direction along one circular arc with a focus of the X-ray tube 11 serving as the center, for example.

The X-ray detector 12 has a structure in which the plurality of detector element arrays is arranged in a slice direction (row direction), for example. There are various types of X-ray CT apparatus 1, such as a Rotate/Rotate-Type (third generation CT) in which the X-ray tube 11 and the X-ray detector 12 rotate together around the subject P, and a Stationary/Rotate-Type (fourth generation CT) in which a large number of X-ray detection elements arrayed in a ring form are fixed and only the X-ray tube 11 rotates around the subject P. Any of these types can be applied to the present exemplary embodiment.

The X-ray detector 12 is an indirect conversion-type detector having a grid, a scintillator array, and an optical sensor array, for example. The scintillator array has a plurality of scintillators, and the scintillators have scintillator crystals that output light with a photon amount corresponding to the amount of incident X-rays. The grid is arranged on the X-ray incident side surface of the scintillator array, and has an X-ray shielding plate that has a function of absorbing scattered X-rays.

The grid may also be called a collimator (one-dimensional collimator or two-dimensional collimator). The optical sensor array has a function of converting the light from the scintillators into an electrical signal according to the amount of light, and has optical sensors, such as photomultiplier tubes (PMTs). The X-ray detector 12 may be a direct conversion-type detector having a semiconductor element that converts the incident X-rays into an electrical signal. The X-ray detector 12 may also be a photon counting-type X-ray detector. The X-ray detector 12 is an example of an X-ray detection unit.

The rotational frame 13 is an annular frame that supports the X-ray tube 11 and the X-ray detector 12 to face each other and rotates the X-ray tube 11 and the X-ray detector 12 using the control device 15 described below. In addition to the X-ray tube 11 and the X-ray detector 12, the rotational frame 13 further includes and supports the X-ray high-voltage device 14 and the DAS 18.

The rotational frame 13 is rotatably supported by a non-rotational portion (e.g., a fixed frame, not illustrated in FIG. 1) of the gantry device 10. A rotational mechanism includes a motor that generates a rotational driving force, and a bearing that transmits the rotational driving force to the rotational frame 13 to rotate the rotational frame 13, for example. The motor is disposed in the non-rotational portion, for example, and the bearing is physically connected to the rotational frame 13 and the motor, and the rotational frame 13 rotates in response to the rotational force of the motor.

The rotational frame 13 and the non-rotational portion each include non-contact or contact-type communication circuitry, which allows communication between a unit supported by the rotational frame 13 and the non-rotational portion or communication between the unit and a device outside of the gantry device 10. For example, when optical communication is adopted as a non-contact communication method, the detection data generated by the DAS 18 is transmitted through optical communication, from a transmitter that includes light-emitting diodes (LEDs) and is disposed on the rotational frame 13 to a receiver with photodiodes disposed on the non-rotational portion of the gantry device 10, and is further transferred from the non-rotational portion to the console device 40 by the transmitter.

As another communication method, a non-contact data transmission method such as a capacitive coupling type or an electromagnetic wave type may be adopted, or a contact-type data transmission method using slip rings and electrode brushes may be adopted. The rotational frame 13 is an example of a rotational unit.

The X-ray high-voltage device 14 has electric circuitry, such as a transformer and a rectifier, and includes a high-voltage generator having a function of generating a high voltage to be applied to the X-ray tube 11 and a filament current to be supplied to the X-ray tube 11, and an X-ray control device that controls an output voltage according to the X-rays emitted by the X-ray tube 11. The high-voltage generator may be of a transformer type or an inverter type. The X-ray high-voltage device 14 may be provided on the rotational frame 13, or may be provided on the fixed frame side of the gantry device 10. The X-ray high-voltage device 14 is an example of an X-ray high-voltage unit.

The control device 15 has processing circuitry having a central processing unit (CPU) and the like, and a driving mechanism such as a motor and an actuator. The control device 15 has a function of receiving an input signal from the console device 40 or an input interface attached to the gantry device 10 and controlling the operations of the gantry device 10 and the patient table device 30.

For example, the control device 15 receives an input signal and performs a control to rotate the rotational frame 13, a control for tilting the gantry device 10, and a control for operating the patient table device 30 and a top plate 33. The control device 15 performs a control to tilt the gantry device 10 by rotating the rotational frame 13 around an axis parallel to the X-axis direction, based on inclination angle (tilt angle) information input through the input interface attached to the gantry device 10.

The control device 15 may be provided in the gantry device 10 or in the console device 40. The control device 15 may be configured to directly install a program into the circuitry of the processor instead of storing the program in the memory. The control device 15 is an example of a control unit.

The wedge 16 is a filter for adjusting the amount of X-rays emitted from the X-ray tube 11. Specifically, the wedge 16 is a filter through which the X-rays emitted from the X-ray tube 11 transmits and attenuates so that the X-rays from the X-ray tube 11 is applied to the subject P with a predetermined distribution. The wedge 16 is a wedge filter or a bow-tie filter, for example, and is made of aluminum processed to have a predetermined target angle and a predetermined thickness.

The collimator 17 is a lead plate or the like used to limit the X-rays that have passed through the wedge 16 into an X-ray irradiation range, and has a slit formed by combining a plurality of lead plates or the like. The collimator 17 may also be called an X-ray limiter.

The DAS 18 has an amplifier that amplifies the electrical signals output from the X-ray detection elements of the X-ray detector 12, and an analog-to-digital (A/D) converter that converts the electrical signals into digital signals, and generates detection data. The detection data generated by the DAS 18 is transferred to the processing circuitry 44. The detection data may also be called pure raw data. The DAS 18 is an example of a data collection unit.

The patient table device 30 is a device on which the subject P to be scanned is placed and moved, and includes a base 31, a patient table driving device 32, the top plate 33, and a support frame 34. The base 31 is a housing that supports the support frame 34 to be movable in the vertical direction. The patient table driving device 32 is a motor or an actuator that moves the top plate 33 on which the subject P is placed, in the longitudinal direction of the top plate 33. The top plate 33, which is provided on the upper surface of the support frame 34, is a plate on which the subject P is to be placed. The patient table driving device 32 may move, in addition to the top plate 33, the support frame 34 in the longitudinal direction of the top plate 33.

The console device 40 has a memory 41, a display 42, an input interface 43, and the processing circuitry 44. Data communication among the memory 41, the display 42, the input interface 43, and the processing circuitry 44 is performed via a bus, for example. Although the console device 40 is described as separate from the gantry device 10, the gantry device 10 may include the console device 40 or some of the components of the console device 40.

The memory 41 is implemented by a semiconductor memory element, such as a random access memory (RAM) or a flash memory, a hard disk, an optical disk, a solid state drive (SSD), and the like, for example. The memory 41 stores detection data output from the DAS 18, projection data generated by a pre-processing function 442, medical image data reconstructed by a reconstruction processing function 443, data of images processed by an image processing function 444, and imaging conditions related to the scan of the subject P, for example.

The medical image data is three-dimensional CT image data, for example, and is also called reconstructed image data or volume data. The data before pre-processing performed by the pre-processing function 442 (detection data or pure raw data) and the projection data is collectively called raw data. That is, the raw data may be pure raw data or projection data.

The memory 41 stores programs related to the execution of a system control function 441, the pre-processing function 442, the reconstruction processing function 443, and the image processing function 444, all of which are executed by the processing circuitry 44. The memory 41 is an example of a storage unit.

The display 42 displays various types of information. For example, the display 42 outputs medical images (CT images) generated by the processing circuitry 44, a graphical user interface (GUI) for receiving various operations from an operator, such as setting of imaging conditions and reconstruction retries, and the like.

For example, the display 42 may be a liquid crystal display (LCD), a cathode ray tube (CRT) display, an organic electroluminescence display (OELD), a plasma display, or any other display, as appropriate.

The display 42 may be provided in the gantry device 10. The display 42 may be of a desktop type, or may be configured with a tablet terminal or the like that is wirelessly communicable with the main body of the console device 40. The display 42 is an example of a display unit.

The input interface 43 receives various input operations from the operator, converts the received input operations into electrical signals, and outputs the electrical signals to the processing circuitry 44. For example, the input interface 43 receives, from the operator, imaging conditions for collecting projection data, reconstruction conditions for reconstructing CT image data, image processing conditions related to post-processing to be performed on CT image data, and the like.

The post-processing may be performed by either the console device 40 or an external workstation. Alternatively, the post-processing may be performed simultaneously by both the console device 40 and the workstation. The post-processing here is defined as a concept that refers to processing to be performed on the image reconstructed by the reconstruction processing function 443.

For example, the post-processing includes multi planar reconstruction (MPR) display of medical images, rendering of volume data, and the like. As the input interface 43, a mouse, a keyboard, a trackball, a switch, a button, a joystick, a touch pad, a touch panel display, and the like can be used as appropriate, for example.

In the present exemplary embodiment, the input interface 43 is not limited to one equipped with physical operation parts, such as a mouse, a keyboard, a trackball, a switch, a button, a joystick, a touch pad, and a touch panel display. Examples of the input interface 43 include also electrical signal processing circuitry that receives an electrical signal corresponding to an input operation from an external input device provided separately from the console device 40 and outputs the electrical signal to the processing circuitry 44.

The input interface 43 may be provided in the gantry device 10. The input interface 43 may be configured with a tablet terminal or the like that is wirelessly communicable with the main body of the console device 40. The input interface 43 is an example of an input unit.

The processing circuitry 44 controls the operation of the entire X-ray CT apparatus 1 in response to an electrical signal of an input operation output from the input interface 43, for example. For example, the processing circuitry 44 has, as hardware resources, processors, such as a CPU, a micro processing unit (MPU), and a graphics processing unit (GPU), and memories, such as a read only memory (ROM) and a random access memory (RAM).

The processing circuitry 44 executes the system control function 441, the pre-processing function 442, the reconstruction processing function 443, and the image processing function 444 by the processor executing programs developed in the memory 41. Each of the functions 441 to 444 is not limited to being implemented by a single piece of processing circuitry. A plurality of independent processors may be combined into processing circuitry, and the functions 441 to 444 may be implemented by the corresponding processors executing programs.

The system control function 441 controls each function of the processing circuitry 44 based on an input operation from the operator via the input interface 43. The system control function 441 also reads out a control program from the memory 41, develops the same on the memory in the processing circuitry 44, and controls each unit of the X-ray CT apparatus 1 in accordance with the developed control program. The system control function 441 is an example of a control unit.

The pre-processing function 442 generates data by subjecting the detection data output from the DAS 18 to pre-processing, such as logarithmic conversion, offset correction, inter-channel sensitivity correction, and beam hardening correction. As described above, data before the pre-processing is called pure raw data, and data having been subjected to the pre-processing is called projection data. The pre-processing function 442 is an example of a pre-processing unit.

The reconstruction processing function 443 performs reconstruction processing on raw data generated by scanning the subject P, thus reconstructing a medical image. Specifically, the reconstruction processing function 443 performs reconstruction processing using a filtered back projection method (FBP method) or the like on the projection data generated by the pre-processing function 442, thus generating data of the medical image.

The reconstruction processing includes various types of processing, such as various corrections such as scattering correction and beam hardening correction, and application of a reconstruction function in the reconstruction conditions. The reconstruction processing function 443 stores the data of the reconstructed medical image in the memory 41. The reconstruction processing function 443 is an example of a reconstruction processing unit.

The image processing function 444 converts the medical image data into tomographic image data on a cross section or three-dimensional image data with a known method, in response to an input operation received from the operator via the input interface 43. The three-dimensional image data may be generated directly by the reconstruction processing function 443. The image processing function 444 is an example of an image processing unit.

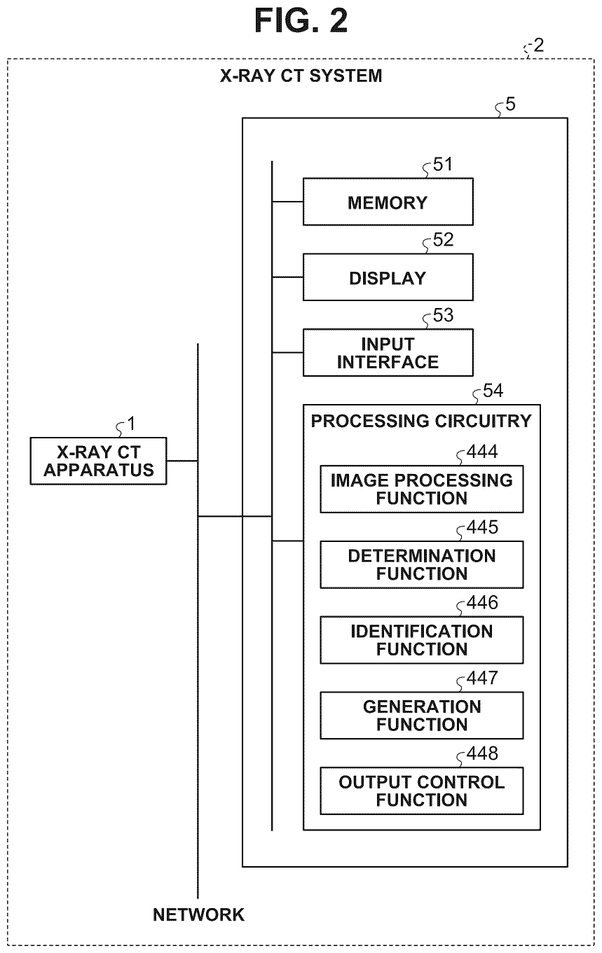

FIG. 2 is a diagram illustrating an example of a configuration of an X-ray CT system 2 according to the exemplary embodiment. As illustrated in FIG. 2, the X-ray CT system 2 includes the X-ray CT apparatus 1 and a medical image processing apparatus 5. The medical image processing apparatus 5 may be incorporated into a medical image management system (hereinafter, referred to as picture archiving and communication systems (PACS)) or a hospital system (hereinafter, referred to as hospital information system (HIS)), or may be implemented as a terminal device connected to a PACS server or an HIS server, for example. For example, the medical image processing apparatus 5 may function as an interpretation terminal.

The medical image processing apparatus 5 has a memory 51, a display 52, an input interface 53, and processing circuitry 54. Data communication among the memory 51, the display 52, the input interface 53, and the processing circuitry 54 is performed via a bus, for example. The hardware configurations of the memory 51, the display 52, the input interface 53, and the processing circuitry 54 are similar to those of the memory 41, the display 42, the input interface 43, and the processing circuitry 44 in the X-ray CT apparatus 1, respectively, and thus description thereof is omitted. Details of the processing of the image processing function 444 of the processing circuitry 44 are similar to those of the X-ray CT apparatus 1.

The memory 51 stores a plurality of medical images generated by the X-ray CT apparatus 1. The plurality of medical images is accompanied by supplementary information, such as version information for the software in the X-ray CT apparatus 1 related to the generation of the medical images, the model number of the X-ray CT apparatus 1, and the name of the X-ray CT apparatus 1. The memory 51 is an example of a storage device.

The processing circuitry 54 executes an image processing function 444, a determination function 445, an identification function 446, a generation function 447, and an output control function 448 by a processor executing programs developed in memory. Each of the functions 444 to 448 is not limited to being implemented by a single piece of processing circuitry. A plurality of independent processors may be combined into processing circuitry, and the functions 444 to 448 may be implemented by the corresponding processors executing programs.

The determination function 445 determines the imaging conditions for imaging the subject P based on an examination order output from a radiology information system (RIS) or an HIS. Specifically, the determination function 445 determines the imaging conditions in accordance with an examination order based on an instruction from the operator via the input interface 53. In determining the imaging conditions, the body posture, body position, and the like of the subject P during a scan may be input with an instruction from the operator via the input interface 53.

If the body posture, body position, and the like of the subject P during a scan are described in the examination order, the determination function 445 may determine the body position of the subject P during the scan using the body posture, body position, and the like described in the examination order. At this time, the display 52 displays the determined imaging conditions together with the information about the body position in the examination order.

The determination function 445 determines the body position of the subject P during a scan based on the imaging conditions related to the scan of the subject P. Examples of the body position of the subject P during a scan include a standing position, a sitting position, and a lying position.

Specifically, in a case where the scan performed under imaging conditions is a scan in a standing position (hereinafter, referred to as a standing scan), the determination function 445 determines that the body position of the subject P is a standing position. In a case where the scan performed under imaging conditions is a scan in a lying position (hereinafter referred to as a lying scan), the determination function 445 determines that the body position of the subject P is a lying position. In a case where the scan performed under imaging conditions is a scan in a sitting position (hereinafter referred to as a sitting scan), the determination function 445 determines that the body position of the subject P is a sitting position.

Imaging conditions include settings for each scan, such as dose, tube voltage, tube current, scan speed, slice thickness, and imaging mode, phases indicating the imaging order for performing a pre-scan for setting the imaging range and a main scan such as a helical scan or step-and-shoot scan. The imaging conditions are also referred to as imaging protocols.

The imaging mode corresponds to various scan modes, such as helical scan (H), step-and-shoot scan (S & S), scan-and-view (S & V), and dynamic scan. The imaging conditions may include body posture information described in the examination order.

The determination function 445 may determine the imaging conditions and the above-described body posture based on an instruction from the operator via the input interface 43. The determination function 445 is an example of a determination unit. The determination function 445 may estimate the body posture of the subject P during a scan by comparing combinations of various items related to the body posture in the imaging conditions with a correspondence table of body postures and the combinations.

The identification function 446 identifies a body position image corresponding to the body position of the subject P. More specifically, the identification function 446 identifies a body position image corresponding to the body position of the subject P determined by the determination function 445, from the memory 51 that stores body position images each representing a body position in association with the corresponding type of the body position. The identification function 446 is an example of an identification unit.

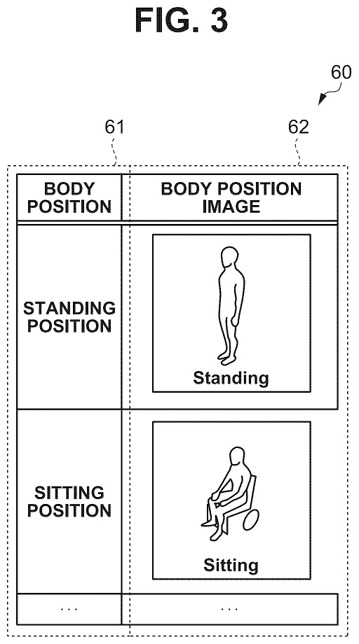

The memory 51 stores body position images each representing a body position in association with the corresponding type of the body position. The information in which body position images representing body positions are in association with the corresponding types of the body positions is called body position information. The body position information here includes the name of the body position and the body position image corresponding to the body position. The body position information will be described with reference to FIG. 3.

FIG. 3 is a schematic diagram illustrating an example of body position information 60 according to the exemplary embodiment. As illustrated in FIG. 3, the body position information includes a body position 61 and a body position image 62. The body position 61 is identification information for identifying each body position. For example, the body position 61 includes information indicating the name of the body position. The body position image 62 is image data representing the body position corresponding to the body position 61. For example, the body position image 62 is an icon that schematically indicates the name of the body position 61. FIG. 3 illustrates icons indicating the standing and sitting positions of the body position 61, and the standing and sitting states.

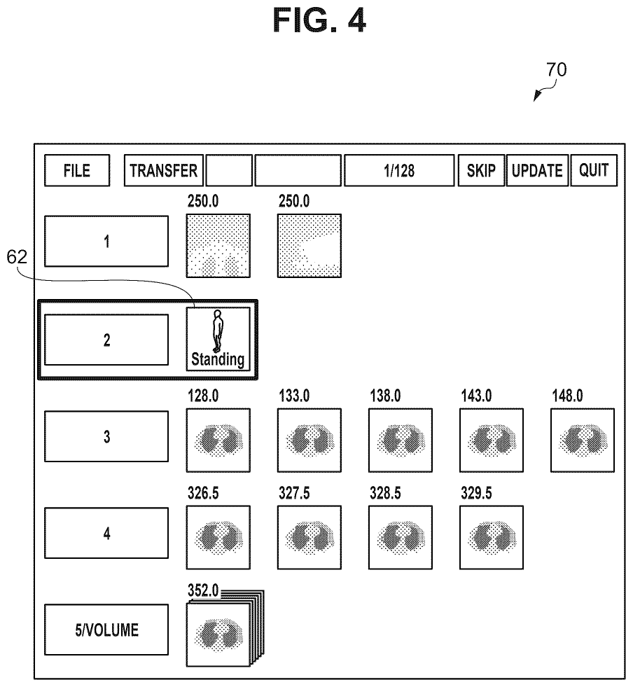

Returning to FIG. 2, the generation function 447 generates subject information about the subject P in which a medical image reconstructed by scanning is in association with a body position image. Specifically, the generation function 447 generates subject information about the subject P in which a medical image reconstructed by the reconstruction processing function 443 is in association with a body position image identified by the identification function 446. The generation function 447 is an example of a generation unit. The subject information will be described with reference to FIG. 4.

FIG. 4 is a schematic diagram illustrating an example of subject information according to the exemplary embodiment. In FIG. 4, subject information 70 includes a plurality of medical images reconstructed by the reconstruction processing function 443 and a body position image 62. The plurality of medical images is schematically arranged by series or volume, for example.

The subject information illustrated in FIG. 4 is generated by the generation function 447 arranging the plurality of medical images and the body position image in a similar manner so that the body position image can be handled similarly to the plurality of medical images.

Returning to FIG. 2, the output control function 448 outputs the subject information. Specifically, the output control function 448 outputs the subject information generated by the generation function 447 to the display 52. The output control function 448 is an example of an output control unit. Accordingly, the output control function 448 displays information in which a medical image generated by a scan is in association with the body position image 62 related to the body posture of the subject during the scan. The output control function 448 also displays the body position image 62 as an icon. A doctor can check the subject information output by the output control function 448 to more easily understand the body position and imaging conditions during imaging than with typical methods.

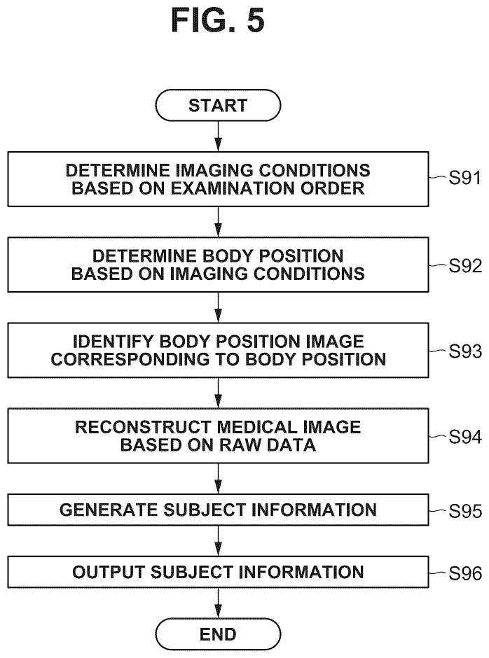

FIG. 5 is a flowchart illustrating an example of a control process of the medical image processing apparatus 5 according to the exemplary embodiment. Initially, in step S91, the determination function 445 determines imaging conditions for the subject P based on an examination order output from the RIS or HIS. Then in step S92, the determination function 445 determines the body position of the subject P during a scan based on the imaging conditions related to the scan of the subject P determined in step S301.

Subsequently, in step S93, the identification function 446 identifies a body position image corresponding to the body position of the subject P determined by the determination function 445 from the memory 51 that stores body position images each representing a body position in association with the corresponding type of the body position. Then in step S94, the reconstruction processing function 443 executes a reconstruction process on the raw data that has been generated by scanning the subject P and transferred from the X-ray CT apparatus 1, thus reconstructing a medical image.

In step S95, the generation function 447 generates subject information about the subject P in which the medical image reconstructed by the reconstruction processing function 443 is in association with the body position image identified by the identification function 446, from the body position information identified by the identification function 446. In step S96, the output control function 448 outputs the subject information generated by the generation function 447 to the display 52. Upon completion of this step, the process by the processing circuitry 54 of the medical image processing apparatus 5 is ended.

As described above, according to the exemplary embodiment, the medical image processing apparatus 5 determines the body position of the subject P during a scan based on the imaging conditions related to the scan of the subject P, identifies a body position image corresponding to the determined body position of the subject P from the storage device that stores body position images each representing a body position in association with the corresponding type of the body position, generates subject information about the subject P in which the medical image obtained by the scan is in association with the identified body position image, and outputs the subject information.

This enables the medical image processing apparatus 5 to display information in which a medical image generated by a scan is in association with a body position image corresponding to the body position of the subject during the scan. In addition, even in a case where a doctor checks subject information that includes a medical image having been transferred to an external device outside the X-ray CT system 2, for example, the medical image is in association with a body position image in the subject information, so that the doctor can easily determine the imaging mode in the medical image and more easily understand the body position of the subject during imaging and imaging situation than with typical methods.

The above-described exemplary embodiment can be modified as appropriate by changing some of the components or functions of each device. Therefore, some modifications of the above-described exemplary embodiment will be described below as other exemplary embodiments. Hereinafter, the differences from the above-described exemplary embodiment will be mainly described, and the matters in common with the above-described exemplary embodiment will be denoted with the same reference numerals and detailed description thereof will be omitted. The other exemplary embodiments described below may be implemented individually or in combination as appropriate.

### First Modification

The image processing function 444, the determination function 445, the identification function 446, the generation function 447, and the output control function 448 in processing circuitry 54 described above in the exemplary embodiment are not limited to the processing circuitry of the medical image processing apparatus 5, and may be implemented by the processing circuitry 44 of the console device 40. The details of the processing to be performed by the image processing function 444, the determination function 445, the identification function 446, the generation function 447, and the output control function 448 in the processing circuitry 44 are similar to those in the exemplary embodiment, and thus description thereof is omitted.

### Second Modification

In the above-described exemplary embodiment, the body positions of the subject P have been described as standing position, sitting position, lying position, and the like, but the body positions of the subject P are not limited to them. For example, the lying position of the subject P may include supine position, lateral position, prone position, and the like.

For example, in the X-ray CT apparatus 1, lying-position imaging is mostly performed in the supine position, but imaging may also be performed in the lateral or prone position. In such cases, the direction of gravity with respect to the subject P changes, so that the doctor is to correctly grasp the body position information before making a diagnosis. For this reason, the identification function 446 of the medical image processing apparatus 5 may generate body position images corresponding to the supine, lateral, and prone positions and store them in a memory 51. That is, the body position images include images corresponding to the standing position, sitting position, supine position, lateral position, and prone position.

There are also imaging modes in which the imaging location changes, as in a two-room CT system. Even in this case, since the shape of the patient table device changes, such as a CT patient table device or an angiography patient table device, the identification function 446 of the X-ray CT apparatus 1 may generate an imaging mode image linked to imaging conditions in association with the body position image, for a display of the imaging situation, for example, and store the imaging mode image in the memory 41.

### Third Modification

For example, in an X-ray CT examination, imaging may be performed with synchronization of signals from medical devices that measure the state of a subject, such as an injector, an electrocardiograph, and a respirometer. Even in such a case, in order to indicate the imaging situation, the identification function 446 of the X-ray CT apparatus 1 may generate an imaging mode image that illustrates an external device in combination with a body position image and store the imaging mode image in the memory 41. That is, the body position image includes an image corresponding to an external device that is used during a scan to measure the state of the subject.

### Fourth Modification

For example, an identification function 446 of an X-ray CT apparatus 1 can determine the body position and imaging conditions of a subject P from information such as the position coordinates, body thickness, and supplementary information for the X-ray CT apparatus 1 that can be acquired from raw data generated by scanning the subject P, and external signals embedded in other raw data (e.g., electrocardiogram information, respiratory information, and the like), and can generate an imaging mode image corresponding to the imaging conditions in conjunction with image reconstruction. The position coordinates of the X-ray CT apparatus 1 are internal information about mechanical angle detection performed with an acceleration sensor or the like provided in the gantry device 10, or positioning imaging (positioning scan) of the subject P.

The identification function 446 can also generate an imaging mode image corresponding to the imaging conditions based on raw data information, not in conjunction with image reconstruction. That is, the identification function 446 of the medical image processing apparatus 5 may identify a body position image based on output information (raw data information) output by an external device that measures the state of the subject P during a scan. The external device here is a modality such as the X-ray CT apparatus 1.

### Fifth Modification

For example, as a method for storing an imaging body position and/or an imaging mode in an external device (e.g., the medical image processing apparatus 5) as an imaging mode image corresponding to imaging conditions at a time other than during execution of an examination, the identification function 446 of the X-ray CT apparatus 1 may generate an imaging mode image in conjunction with the transfer of a medical image to the external device and transfer the imaging mode image to the external device.

### Sixth Modification

For example, the medical image processing apparatus 5 may generate a body position image. In such a case, the identification function 446 of the medical image processing apparatus 5 may generate an imaging mode image indicating an imaging body position and/or an imaging mode, based on information obtained from an image at the time when the medical image processing apparatus 5 receives medical image data, and store the imaging mode image in the memory 41. That is, the identification function 446 of the medical image processing apparatus 5 may identify a body position image based on a medical image.

### Seventh Modification

For example, the generation function 447 of the medical image processing apparatus 5 may associate a body position image with a part of an examination summary. Specifically, the generation function 447 may generate medical information in which a body position image is in association with information about a subject examination summary that summarizes examinations performed on a subject P at a plurality of time points.

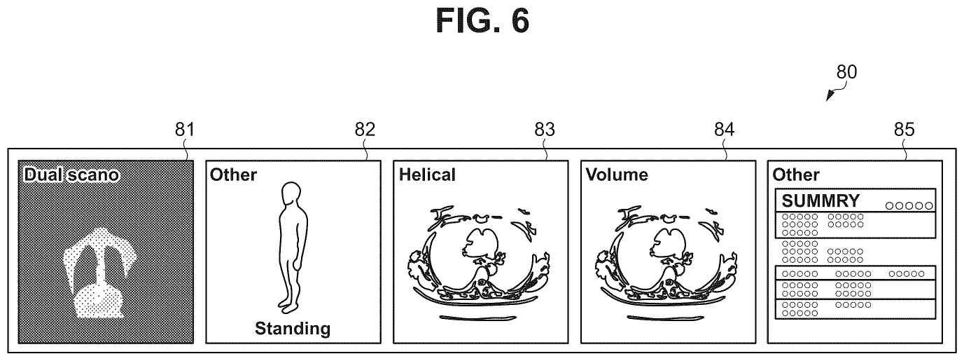

FIG. 6 is a diagram illustrating an example of medical information according to a seventh modification. Medical information 80 illustrated in FIG. 6 provides a plurality of medical images 81, 83, and 84, a body position image 82, and a subject examination summary 85 that summarizes examinations performed at a plurality of time points. This allows a doctor to view the medical images and the body position image together with the examination summary.

### Eighth Modification

For example, the X-ray CT apparatus 1 or the generation function 447 of the medical image processing apparatus 5 may embed information about an imaging mode indicating the imaging mode in a reconstructed medical image. This is different from supplemental information. The embedment means generation of a medical image in which a specific image and/or a character string are/is embedded in a stamp-like manner. That is, the generation function 447 may generate a superimposed medical image by superimposing a body position image on the medical image. The generation function 447 may generate information indicating the body position corresponding to a body position image as supplementary information.

### Ninth Modification

For example, the X-ray CT apparatus 1 or the identification function 446 of the medical image processing apparatus 5 may automatically identify the body posture of a subject P based on output information from external devices such as various cameras provided in the examination room in which the X-ray CT apparatus 1 is installed.

For example, in a case where an external device such as a camera is used, it is possible to recognize the imaging situation and the silhouette of a patient from the camera image and automatically generate an imaging mode image. In such a case, it is also possible to register, as an image, the imaging mode image even for a special body position or imaging situation that is not stored in advance in the memory 51. In addition, the camera image may be registered as it is without creating an imaging mode image. That is, the identification function 446 of the medical image processing apparatus 5 may identify a body position image from captured images of the state of the subject P during a scan.

The identification function 446 may make a determination from specific information such as imaging conditions, use image data stored in advance in the memory 51, and store the image data in the memory 51 as an imaging mode image. The identification function 446 may also determine imaging conditions using an external device such as a camera, determine image data that matches the image data stored in advance in the memory 51, and store the image data in the memory 51 as an imaging mode image.

According to at least one of the exemplary embodiments described above, it is possible to display information in which a medical image generated by a scan is in association with information about the body posture of the subject during the scan.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus, comprising processing circuitry configured to:
determine a body position of a subject during a scan based on an imaging condition related to the scan of the subject;
identify, from a storage device that stores a body position image representing the body position in association with a corresponding type of the body position, a body position image corresponding to the determined body position of the subject;
generate subject information about the subject in which a medical image obtained by the scan is in association with the identified body position image; and
output the generated subject information.

2. The medical image processing apparatus according to claim 1, wherein the body position image is identified from output information output by an external device that scans the subject.

3. The medical image processing apparatus according to claim 1, wherein the body position image is identified based on the medical image.

4. The medical image processing apparatus according to claim 1, wherein the body position image is identified from a captured image of a state of the subject during the scan.

5. The medical image processing apparatus according to claim 1, wherein information in which the body position image is in association with medical information about a subject examination summary that summarizes examinations performed on the subject at a plurality of time points is generated.

6. The medical image processing apparatus according to claim 1, wherein a superimposed medical image obtained by superimposing the body position image on the medical image is generated.

7. The medical image processing apparatus according to any one of claim 1, wherein the body position image is prepared for each of body position types including a standing position, a sitting position, a supine position, a lateral position, and a prone position.

8. The medical image processing apparatus according to claim 7, wherein the body position image includes an image corresponding to an external device that is used during the scan to measure the state of the subject.

9. The medical image processing apparatus according to claim 1, wherein the body position image is displayed as an icon.

10. An X-ray computed tomography apparatus, comprising processing circuitry configured to:
perform a reconstruction process on raw data generated by scanning a subject to reconstruct a medical image;
determine a body position of a subject during a scan based on an imaging condition related to the scan of the subject;
identify, from a storage device that stores a body position image representing the body position in association with a corresponding type of the body position, a body position image corresponding to the determined body position of the subject;
generate subject information about the subject in which a medical image obtained by the scan is in association with the identified body position image; and
output the generated subject information.
